(19) United States
(12) Patent Application Publication
(10) Pub. No.: US 2021/0122926 A1
(43) Pub. Date: Apr. 29, 2021

(54) PROTECTION OF SURFACES BY EVAPORATED SALT COATINGS

(71) Applicant: NANOXCOATINGS LC, Salt Lake City, UT (US)

(72) Inventors: Matthew R. Linford, Orem, UT (US); Dhruv Shah, Provo, UT (US)

(21) Appl. No.: 17/084,290

(22) Filed: Oct. 29, 2020

Related U.S. Application Data
(60) Provisional application No. 62/927,583, filed on Oct. 29, 2019.
(60) Provisional application No. 62/957,055, filed on Jan. 3, 2020.
(60) Provisional application No. 63/038,540, filed on Jun. 12, 2020.

(51) Int. Cl. C09D 5/00; C23C 16/04; C23C 16/56; C23C 16/40; A61L 31/08

(52) U.S. Cl. CPC C09D 5/008; C23C 16/04; A61L 31/082; C23C 16/403; C23C 16/56

(57) ABSTRACT

A method for preventing contamination of a substrate surface includes obtaining a substrate having a surface to be protected from contamination and depositing a removable protective salt coating on the substrate surface. A disclosed method also includes storing the substrate surface having the removable protective salt coating for a time period and then removing the protective salt coating. A method for selectively preventing atomic layer deposition (ALD) on a substrate surface exposed to an ALD process includes depositing a removable protective salt coating on the substrate surface, exposing the surface to an ALD process, and removing the protective salt coating. Some disclosed substrate surfaces include a thiol-on-gold monolayer, a silicon wafer, glass, a silanized surface, and a dental implant. The protective salt coating may have a thickness in the range of 50 nm to 1 μm. The protective salt coating may be deposited by thermal evaporation or similar process.

### CROSS-REFERENCE TO RELATED APPLICATIONS

This application claims the benefit of U.S. Provisional Application No. 62/927,583, filed Oct. 29, 2019 and entitled “PROTECTION OF SURFACES BY EVAPORATED SALT FILMS.” This application claims the benefit of U.S. Provisional Application No. 62/957,055, filed Jan. 3, 2020 and entitled “PROTECTION OF SURFACES BY EVAPORATED SALT FILMS.” This application claims the benefit of U.S. Provisional Application No. 63/038,540, filed Jun. 12, 2020 and entitled “PROTECTION OF THIOL-ON-GOLD MONOLAYERS USING DISSOLVABLE SALT FILMS.” The foregoing applications are incorporated herein by reference in their entirety for all purposes.

### BACKGROUND OF THE INVENTION

The invention relates to methods for preventing or limiting surface contamination and/or for storing surfaces/substrates such that contamination on them is limited. The disclosed methods deposit a salt coating or salt film on a surface to be protected, which can be removed at a later date and return the surface to its clean or pre-coated condition.

Surface contamination can limit the performance and applications of surfaces. Surface contamination can decrease surface reactivity. Compared to a pristine surface, a contaminated surface may show poor adhesion of thin coatings or other materials. For instance, it may be difficult to construct a microfabricated circuit or to deposit a thin coating on a dirty surface or substrate. Surface contamination changes surface wetting, which impacts many industrially relevant processes. Surface contamination may react with limit the reactivity of reactive surfaces. Removal of surface contaminants adds additional steps to a process. Surface contamination often requires additional surface cleaning, which in some cases can result in additional surface contamination.

It would be an advancement in the art to protect surfaces from contamination. It would be a further advancement in the art to protect reactive surfaces from unintended reaction. It would be yet another advancement in the art to provide a simple method for substrate protection and deprotection.

The subject matter disclosed and claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to methods for preventing contamination of a substrate surface. Various aspects and embodiments within the scope of the disclosed invention are listed below. It will be understood that the embodiments listed below may be combined not only as listed below, but in other suitable combinations in accordance with the scope of the invention.

One aspect of the disclosed method involves obtaining a substrate having a surface to be protected from contamination and depositing a removable protective salt coating on the substrate surface. The disclosed method for preventing surface contamination of a substrate surface may further include storing the substrate surface having the removable protective salt coating for a time period. The present disclosure also relates to substrate surfaces that are protected from contamination by a removable protective salt coating deposited on the substrate surface.

Non-limiting examples of surfaces to be protected from contamination include clean surfaces. Clean surfaces include a surface which spontaneously becomes contaminated when exposed to ambient atmospheric conditions. Exposure to ambient atmospheric conditions, including air, moisture, dust, or reactive chemical compounds present in the atmosphere, may spontaneously contaminate a surface.

Non-limiting examples of surfaces to be protected from contamination also include surfaces which contain reactive functional groups bound to the surfaces. Reactive functional groups include, but are not limited to, reactive molecular or atomic species bound to the surface. Non-limiting examples of surfaces to be protected from contamination include surfaces which contain biologically active molecules. Biologically molecules include, but are not limited to, amino acids, peptides, proteins, nucleic acids, DNA, and RNA.

Another non-limiting example of a surface to be protected from contamination includes a thiol-on-gold monolayer. The thiol-on-gold monolayer surface may be included in a biosensor. The thiol-on-gold monolayer surface may comprise an alkyl thiol. The thiol-on-gold monolayer surface may comprise a thiol connected to a biologically active molecule.

Yet another non-limiting example of a surface to be protected from contamination includes chemically derivatized surfaces, such as surfaces having a silane coating and surfaces having a silane coating further derivatized with a biologically active molecule.

Additional surfaces to be protected from contamination include, but are not limited to, silicon wafers, glass, dental materials such as crowns and dental implants, and substrate surfaces used in coated blade spray (CBS) and solid-phase microextraction (SPME) technologies.

As used herein, the word “salt” means any ionic compound, inorganic or organic. As used herein, the term “salt coating” means a continuous coating or layer of salt deposited on a designated substrate surface.

The protective salt coating disclosed herein can vary in thickness from about a nanometer to hundreds of nanometers. In some embodiments, the protective salt coating may have a thickness in the range of 50 nm to 1 μm. In some embodiments, the protective salt coating may have a thickness in the range of 50 nm to 200 nm.

It is within the scope of the disclosed invention to deposit more than one type of removable protective salt coating on a substrate surface. For example, a substrate surface may be protected with a layer of NaCl, a second layer of KBr, and a third layer of NaCl.

One application for the removable protective salt coatings disclosed herein is as a protective layer in atomic layer deposition (ALD). A removable protective salt coating is be deposited on one side of a substrate. ALD is performed on the entire substrate. The protective salt coating is washed or rinsed away, which can be designed to remove undesired ALD deposition on the salt-protected side of the substrate, leaving an intact ALD coating on the unprotected side of the substrate.

Thus, the disclosed invention includes a method for selectively preventing atomic layer deposition (ALD) on a substrate surface exposed to an ALD process. In the method a removable protective salt coating is deposited on the surface to be protected. The surface is exposed to an ALD process. The protective salt coating is removed.

One application for the removable protective salt coatings disclosed herein is as a protective layer for a thiol-on-gold monolayer. The thiol-on-gold monolayer surface may be included in a biosensor. The thiol-on-gold monolayer surface may comprise an alkyl thiol. The thiol-on-gold monolayer surface may comprise a thiol connected to a biologically active molecule. The biologically active molecule may be selected from amino acids, peptides, proteins, nucleic acids, DNA, and RNA. In the disclosed thiol-on-gold monolayer surface protected from contamination, the removable salt coating may have a thickness in the range of 50 nm to 1 μm.

One application for the removable protective salt coatings disclosed herein is as a protective layer for a chemically derivatized surface, such as a surface having a silane coating and a surface having a silane coating further derivatized with a biologically active molecule.

One advantage of the disclosed methods for preventing contamination of a substrate surface includes the ability to “capture” and preserve a surface in its pristine state after formation or cleaning so that the pristine state or a near-pristine state can be used at a future date.

Another application for the removable protective salt coatings disclosed herein is in protecting glass microscope slides and glass cover slips, or fused silica microscope slides and fused silica cover slips. In this embodiment of the disclosed invention, the glass or fused silica substrate may be cleaned first by any of a variety of known cleaning methods, e.g., by plasma cleaning or with piranha solution. A removable protective salt coating may then be deposited on the clean, dry surface before it has had a chance to become contaminated to a significant degree. The surface may then be stored. After storage, the salt coating may be removed by rinsing to expose the protected surface, which may then be used for some application.

Another application for the disclosed invention is in protecting substrates like silicon wafers, glass slides, fused silica slides, and molded or extruded plastics with an evaporated salt coating so that they can be stored for prolonged periods of time under atmospheric conditions or other storage conditions.

Plastic substrates, including sheets, may be protected within the scope of the disclosed invention.

In general, after removal of a protective salt coating with water or another solution, the surface will be dried. Surfaces may be blown dry, for example, with a jet of nitrogen, compressed air, or argon. Surfaces may also be dried by the natural spinning action of a spin coater.

The various liquids that may be used to remove the protective salt coating described herein include, but are not limited to, aqueous solutions containing detergents, salts, and/or alcohols or other organic molecules, e.g., acetone, formic acid, or acetic acid. Pure water or pure organic solvents may also be used, depending on the solubility of the protective salt coating.

The surfaces/substrates that may be protected by the salt coatings disclosed herein may be planar or irregular. They may have regular or irregular cross sections. They may be three-dimensional objects, such 3D printed objects.

The salt coatings that are deposited in this disclosure may be deposited as single layers over a surface or may be deposited through a stencil mask so that the resulting salt coating is patterned.

The protective salt coatings disclosed herein may be used to protect (i) thin protein or peptide coatings on surfaces, (ii) thin DNA or RNA coatings on surfaces, (iii) active elements of biochips or bioarrays that may contain DNA, RNA, proteins, or peptides, (iv) microfluidic devices, (v) bioassays, (vi) 6-well plates, (vii) 24-well plates, (viii) 96-well plates, and (ix) paper-based/supported bioassays. The 6, 24, and 96 well plates (or other plates with other numbers of wells) may be made of plastic or a glass and may be protected with a salt coating before or after chemistry is performed on them. It may be advantageous to coat injection molded plastic parts shortly after they are made.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

In order to describe the manner in which the above-recited and other advantages and features of the disclosed invention can be obtained, a more particular description of the invention briefly described above will be rendered by reference to specific embodiments thereof which are illustrated in the appended drawings. It should be understood that these drawings depict only typical embodiments of the invention and are not to be considered to be limiting of its scope, the invention will be described and explained with additional specificity and detail through the use of the accompanying drawings.

FIG. 1 is a schematic flow diagram for a method for selectively preventing atomic layer deposition (ALD) on a substrate surface exposed to an ALD process. In the process, a removable salt coating is deposited on a substrate surface to be protected from ALD, conventional ALD is performed on the unprotected side of the substrate, and the salt coating on the backside of the substrate is removed, together with any unwanted ALD deposition.

FIG. 2 is a schematic flow diagram for a method for protecting and deprotecting surfaces with removable salt coatings. In the process, a removable salt coating is deposited on a substrate surface prone to contaminate, the coated substrate is stored for a period of time, and the salt coating is removed to reveal the substrate surface available for use.

FIG. 3 shows XPS spectra from 0-350 eV of (upper graph) a ca. 100 nm film of NaCl evaporated onto a fused silica slide, and (lower graph) an uncoated fused silica slide.

FIG. 4 shows XPS spectra from 0-325 eV after ALD deposition of Al₂O₃ via 100 cycles of TMA and water on NaCl—coated fused silica (top), and uncoated fused silica after the same ALD deposition (bottom). The uncoated side of this surface was facing up during the deposition.

FIG. 5 shows XPS spectra from 0-210 eV obtained from a fused silica substrate after (i) protection with NaCl on one side, (ii) ALD of Al₂O₃, and (iii) sonication/rinsing with water.

FIG. 6 shows XPS spectra from 0-210 eV of three different fused silica substrates after coating with 10 (top), 50 (middle), or 100 (bottom) nm of NaCl, ALD of Al₂O₃ (100 TMA/water cycles), and sonication/rinsing with water.

FIG. 7 shows XPS spectra from 0-210 eV of a fused silica substrate after (i) one side was coated with NaCl, (ii) ALD of ZnO, and (iii) deprotection of the substrate by sonication/rinsing with water. Zn is only present on the side of the surface that was not coated with NaCl.

### DETAILED DESCRIPTION OF THE INVENTION

The disclosed invention prevents or limits substrate surface contamination and/or provides substrate surfaces with a removable protective salt coating. The disclosed methods deposit a salt coating or salt film on a surface to be protected, which can be removed at a later date and return the surface to its clean or pre-coated condition. Substrate surfaces with the removable protective salt coating may be stored for a time period. Thereafter the protective salt coating is removed revealing the substrate surface with little or no contamination.

Surface contamination decreases surface reactivity, and the additional steps required to clean substrates increase the time and expense of a process. Hence, the prevention or mitigation of surface contamination is an important task. A common surface contaminant is adventitious carbon, which is present on almost all surfaces stored under ambient conditions. Preferred methods for removing adventitious carbon from inorganic substrates include plasma-cleaning and piranha solution.

One aspect of the disclosed method involves obtaining a substrate having a surface to be protected from contamination and depositing a removable protective salt coating on the substrate surface. The disclosed method for preventing surface contamination of a substrate surface may further include storing the substrate surface having the removable protective salt coating for a time period. The time period may range from days to years. The present disclosure also relates to substrate surfaces that are protected from contamination by a removable protective salt coating deposited on the substrate surface.

Non-limiting examples of surfaces to be protected from contamination include clean surfaces. Clean surfaces include a surface which has undergone a cleaning procedure. Clean surfaces include a newly manufactured or prepared surface which has not been exposed to potential contamination. Clean surfaces include a surface which spontaneously becomes contaminated when exposed to ambient atmospheric conditions. Often exposure to ambient atmospheric conditions, including air, moisture, dust, or reactive chemical compounds present in the atmosphere, may spontaneously contaminate a surface.

Non-limiting examples of surfaces to be protected from contamination also include surfaces which contain reactive functional groups bound to the surfaces. Reactive functional groups include, but are not limited to, reactive molecular or atomic species bound to the surface. Non-limiting examples of surfaces to be protected from contamination include surfaces which contain biologically active molecules. Biologically molecules include, but are not limited to, amino acids, peptides, proteins, nucleic acids, DNA, and RNA.

Another non-limiting example of a surface to be protected from contamination includes a thiol-on-gold monolayer. One problem with thiol-on-gold monolayers is their tendency to oxidize with time. This oxidation is believed to take place at the sulfur atom of the thiol, which weakens the sulfur-gold interaction and ultimately reduces the stability of the monolayer. Removable protective salt coatings may be deposited on thiol-on-gold monolayers to prevent their oxidation.

Thiol-on-gold monolayers have many uses, including in biosensors, on quartz crystal microbalance (QCM) surfaces, and in surface plasmon resonance devices. These monolayers may be protected by salt coatings after they are made, stored for some time, and then deprotected shortly before they are used. The thiol-on-gold monolayers used in devices may include alkyl thiols. The thiol-on-gold monolayers may include thiols connected to biologically active molecules. Non-limiting examples of biologically active molecules include amino acids, peptides, proteins, antibodies, nucleotides, DNA, RNA, or ligands.

Additional surfaces to be protected from contamination include, but are not limited to, silicon wafers, glass, dental materials such as crowns and dental implants, and substrate surfaces used in coated blade spray (CBS) and solid-phase microextraction (SPME) technologies.

Surfaces protected by salt coatings may be (i) cleaned and/or formed in a clean state, (ii) coated with a protective salt layer, (iii) stored for a period of time, and (iv) used in some application without formal deprotection or removal of the salt layer. For example, it may be possible to protect dental implants with a salt coating and then use them directly, where the removal of the salt coating will take place by virtue of it being in a human mouth. As another example, a glass slide might be cleaned, coated with a salt film, and then stored. It might then be used directly by a pathologist as a substrate for a tissue sample without the salt coating first being removed. Again, the use/application of a salt-coated material may result in partial or complete removal of the salt coating when it is used. In the case of the dental implant, it may be found that certain salts actually promote desired tissue growth more than others and are therefore advantageous. That is, certain salt coatings is some applications may both protect a surface from contamination and also have additional benefits that make it advantageous not to remove the salt coatings before they are used.

As used herein, the word salt means any ionic compound, inorganic or organic.

As used herein, the term “salt coating” means a continuous coating or layer of salt deposited on a designated substrate surface. One way in which salt coatings may be deposited on a substrate surface is by thermal evaporation. Salt coatings may also be deposited by electron beam evaporation, sputtering, atomic layer deposition, or even through a liquid phase deposition, e.g., via spin coating.

The protective salt coating disclosed herein can vary in thickness from about a nanometer to hundreds of nanometers. In some embodiments, the protective salt coating may have a thickness in the range of 50 nm to 1 μm. In some embodiments, the protective salt coating may have a thickness in the range of 50 nm to 200 nm.

The coating thickness of the protective salt coatings deposited within the scope of the disclosed invention may be determined by any of a variety of techniques, including spectroscopic ellipsometry, atomic force microscopy (when a step edge has been created in the coatings), scanning electron microscopy (SEM), or during deposition using a quartz crystal microbalance or an in situ ellipsometer.

One salt within the scope of the disclosed invention is NaCl, which is also known as table salt or rock salt. Sodium chloride is particularly preferred because of its low cost, availability, low chemical reactivity, and low toxicity. However, other salts may be preferred in other applications. Not all salts respond equally well to water vapor in the air. That is, some are more hygroscopic and others are less hygroscopic. One skilled in the art will understand that different salts with different properties may be selected for protecting substrates as vapor deposited thin films, where the choice of salts here will depend on how they protect a substrate and the degree to which they are hygroscopic.

Other common salts which may be used within the scope of the disclosed invention include, but are not limited to, KCl, MgCl₂, MgF₂, and KBr.

The cations in the salts of the disclosed invention may be monovalent, as in Na⁺, K⁺, Rb⁺, or Cs⁺ or they may be divalent, as in Mg²⁺, Ca²⁺, or Ba²⁺. The cations may be atomic (as in Na⁺ or Ca²⁺) or molecular, as in NH₄⁺ or CH₃NH₃⁺. Similarly, the anions in the protective salt coatings described herein may be monovalent, as in F−, Cl⁻, Br⁻, I⁻, nitrate (NO₃⁻), or acetate (CH₃COO⁻) or divalent, as in sulfate (SO₄²⁻) or carbonate (CO₃²⁻). Similarly, the anions may be atomic or molecular.

It is within the scope of the disclosed invention to deposit more than one type of removable protective salt coating on a substrate surface. For example, a substrate surface may be protected with a layer of NaCl, a second layer of KBr, and a third layer of NaCl.

One application for the removable protective salt coatings disclosed herein is as a protective layer in atomic layer deposition (ALD). A removable protective salt coating is be deposited on one side of a substrate. ALD is performed on the entire substrate. The protective salt coating is washed or rinsed away, which can be designed to remove undesired ALD deposition on the salt-protected side of the substrate, leaving an intact ALD coating on the unprotected side of the substrate.

Thus, the disclosed invention includes a method for selectively preventing atomic layer deposition (ALD) on a substrate surface exposed to an ALD process. In the method a removable protective salt coating is deposited on the surface to be protected. The surface is exposed to an ALD process. The protective salt coating is removed.

One application for the removable protective salt coatings disclosed herein is as a protective layer for a thiol-on-gold monolayer. The thiol-on-gold monolayer surface may be included in a biosensor. The thiol-on-gold monolayer surface may comprise an alkyl thiol. The thiol-on-gold monolayer surface may comprise a thiol connected to a biologically active molecule. The biologically active molecule may be selected from amino acids, peptides, proteins, nucleic acids, DNA, and RNA. In the disclosed thiol-on-gold monolayer surface protected from contamination, the removable salt coating may have a thickness in the range of 50 nm to 1 μm.

One advantage of the disclosed methods for preventing contamination of a substrate surface includes the ability to “capture” and preserve a surface in its pristine state after formation or cleaning so that the pristine state or a near-pristine state can be used at a future date.

Another application for the removable protective salt coatings disclosed herein is in protecting glass microscope slides and glass cover slips, or fused silica microscope slides and fused silica cover slips. In this embodiment of the disclosed invention, the glass or fused silica substrate may be cleaned first by any of a variety of known cleaning methods, e.g., by plasma cleaning or with piranha solution. A removable protective salt coating may then be deposited on the clean, dry surface before it has had a chance to become contaminated to a significant degree. The surface may then be stored. After storage, the salt coating may be removed by rinsing to expose the protected surface, which may then be used for some application.

Another application for the disclosed invention is in protecting substrates like silicon wafers, glass slides, fused silica slides, and molded or extruded plastics with an evaporated salt coating so that they can be stored for prolonged periods of time under atmospheric conditions or other storage conditions.

An important use of clean glass microscope slides and cover slips is in pathology. That is, pathologists place tissue samples onto cover slips for examination. It is believed that pathologists would benefit from a greater availability of clean glass microscope slides and cover slips. That is, a pathology lab might purchase glass microscope slides that have been protected via the methods/procedures described in this disclosure and then deprotect these slides shortly before using them.

Prior to coating a substrate surface with a removable protective salt coating, the surface may be chemically derivatized. For example, a silicon wafer, a glass or fused silica microscope slide or cover slip, or other substrate surface may be chemically derivatized. One non-limiting example of a chemically derivatized surface includes a silanized surface. Among the many silanes that may be used for this purpose include, but are not limited to, 3-aminopropyltriethoxysilane (APTES), glycidoxypropyltrimethoxysilane (GOPS), and mercaptopropyltriethoxysilane. The silanes may be deposited by gas phase or liquid phase deposition processes. The silane-coated surface may be further derivatized with a biologically active molecule. Biologically molecules include, but are not limited to, amino acids, peptides, proteins, nucleic acids, DNA, and RNA.

A silicon wafer, a glass or fused silica microscope slide or cover slip, or other substrate surface may also be derivatized with a polymer prior to protection with a removable protective salt coating. These polymers may be neutral or polyelectrolytes. These polymers may be deposited by spin coating or adsorption from solution. Possible polymers here include polyethylenimine (PEI) and polyallylamine. These polymers may be deposited as thin coatings only a fraction of a nanometer thick or as thicker coatings, e.g., tens of nanometers thick or thicker. A glass or fused silica microscope slide or cover slip may also be derivatized with an activated ester, e.g., an NETS-ester, a sulfo-NHS-ester, or an acid chloride, and then protected with a salt coating.

A glass or fused silica slide or cover slip may be protected with a removable protective salt coating on one or both of its sides.

Plastic substrates, including sheets, may be protected within the scope of the disclosed invention. Non-limiting examples of plastic substrates include those made from polyethyleneterephthalate (PET), polymethylmethacrylate (PMMA), polystyrene, and other polymers. The protection of these polymeric materials may be done in a roll-to-roll production apparatus that deposits a salt under vacuum. It may also be done immediately after the polymeric material is made, e.g., rolled or extruded. Fibrous materials, e.g., glass fibers or textile (polymeric) fibers may also be coated with removable protective salt coatings disclosed herein. Solid-phase microextraction (SPME) fiber surfaces and coated blade spray (CBS) surfaces may also be coated with removable protective salt coatings disclosed herein.

In general, after removal of a protective salt coating with water or another solution, the surface will be dried. Surfaces may be blown dry, for example, with a jet of nitrogen, compressed air, or argon. Surfaces may also be dried by the natural spinning action of a spin coater.

The various liquids that may be used to remove the protective salt coating described herein include, but are not limited to, aqueous solutions containing detergents, salts, and/or alcohols or other organic molecules, e.g., acetone, formic acid, or acetic acid. Pure water or pure organic solvents may also be used, depending on the solubility of the protective salt coating. More than one rinse step or rinse solution may be used to remove the protective salt coating described herein. The removal of the salt coating may be aided by mechanical agitation, e.g., by use of a brush, or via sonication or heating. The salt coating may also be removed with a spin coater in which the salt-protected surface is loaded on the chuck of the spin coater and spun while water or another rinse solution is directed onto the surface to remove the protective coating. The protective salt coating may be removed by being squirted with water or another solution while it is held with tweezers or while being spun on the chuck of a spin coater. These processes of removing the salt coating (deprotecting the surface) may be automated. In some cases, heat itself may be used to remove a protective coating.

It may be advantageous to deposit a removable protective salt coating that is not highly soluble in water, but that is soluble in an acidic or basic solution. An acidic or basic solution could then be used to remove the salt coating. It may be advantageous here to use a volatile acid or base like aqueous HCl or aqueous NH₃, respectively.

The surfaces/substrates that may be protected by the salt coatings disclosed herein may be planar or irregular. They may have regular or irregular cross sections. They may be three-dimensional objects, such 3D printed objects.

The protective salt coatings that are deposited in this disclosure may be deposited as single layers over a surface or may be deposited through a stencil mask so that the resulting salt coating is patterned.

The protective salt coatings disclosed herein may be used to protect (i) thin protein or peptide coatings on surfaces, (ii) thin DNA or RNA coatings on surfaces, (iii) active elements of biochips or bioarrays that may contain DNA, RNA, proteins, or peptides, (iv) microfluidic devices, (v) bioassays, (vi) 6-well plates, (vii) 24-well plates, (viii) 96-well plates, and (ix) paper-based/supported bioassays. The 6, 24, and 96 well plates (or other plates with other numbers of wells) may be made of plastic or a glass and may be protected with a salt coating before or after chemistry is performed on them. It may be advantageous to coat injection molded plastic parts shortly after they are made.

The ability to coat glass or inorganic surfaces with a removable protective salt coating may be advantageous in various industries. For example, silicon wafers could be cleaned, dried (if necessary), and coated with a salt coating. The salt coating could then be removed at a future time shortly before the surface is used. Similarly, glass surfaces, e.g., display glasses, might be coated with an evaporated salt coating shortly after they are produced. Then, shortly prior to their use as a substrate, e.g., as the substrate for the electronics in a flat panel display, television, or cell phone, the salt coating could be removed. The advantage here is that contamination on the glass surface is limited and subsequent cleaning may then not be necessary, i.e., it may be possible to use the glass substrate directly after deprotection.

The following examples and experimental results are given to illustrate various embodiments within the scope of the present disclosure. These are given by way of example only, and it is understood that the following examples are not comprehensive or exhaustive of the many types of embodiments of the present disclosure that can be prepared in accordance with the present disclosure.

### Example 1

In this example, a thin, thermally-evaporated salt coating was used to protect silicon substrates from ambient/adventitious carbon contamination. In addition, the removable salt coating was used to protect surfaces from unwanted atomic layer deposition (ALD).

ALD is an increasingly important method that provides a high degree of control over thin film growth, and many materials, including metal oxides, nitrides, and sulfides, can be deposited by ALD. Accordingly, ALD is now well accepted in semiconductor manufacturing and nanotechnology. One of the most significant advantages of ALD is that it is a bottom-up approach for adding atoms to a material in a layer-by-layer fashion. ALD uses gas phase precursors, which are often generated from liquids or solids with sufficiently high vapor pressures. The molecular precursor gases used in many ALD experiments are highly reactive and have relatively long mean free paths. Thus, it can be challenging to limit where they might travel and react, i.e., like chemical vapor deposition, ALD is not a line-of-sight technique. Of course, these conditions are advantageous for depositions on irregular substrates, e.g., high aspect ratio structures, powders, and porous materials. However, ALD is limited when selective or spatial deposition is required. That is, there are times when one would wish to perform ALD without coating an entire substrate.

In this example, a high quality, thin film optical standards by ALD was prepared on only one side of fused silica substrates for spectroscopic ellipsometry and transmission UV-VIS studies. The backside of the substrate was protected from unwanted irregular ALD deposition of Al₂O₃ from trimethylaluminum (TMA) and water precursors by depositing a thin removable sodium chloride coating on the backsides of substrates.

Sodium chloride is a common and inexpensive material. It has low toxicity, and it is soluble in water. Evaporation, i.e., sublimation of NaCl, is advantageous because it is a line-of-sight deposition technique. An additional advantage of NaCl is that it is stable under most ALD deposition conditions.

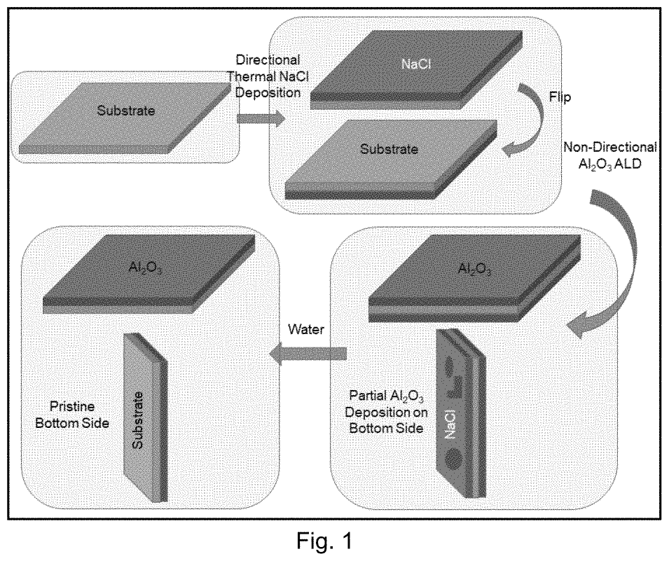

FIG. 1 shows a schematic flow diagram for a method for selectively preventing atomic layer deposition (ALD) on a substrate surface exposed to an ALD process. In the process, a removable salt coating is deposited on a substrate surface to be protected from ALD, conventional ALD is performed on the unprotected side of the substrate, and the salt coating on the backside of the substrate is removed, together with any unwanted ALD deposition.

In this example, a removable sodium chloride salt coating was deposited on one surface of a fused silica substrate by thermal evaporation. Other line-of-sight salt deposition techniques, e.g., pulsed laser deposition, could similarly be useful for depositing protective salt coatings.

After coating with a salt layer, ALD was performed on the substrate with the salt-protected side facing down. After ALD, the surface was rinsed with water, which removed the salt coating and any unwanted ALD deposition on it. NaCl deposition and removal were confirmed by X-ray photoelectron spectroscopy (XPS) and/or spectroscopic ellipsometry (SE). The disclosed method works effectively for the selective thermal ALD deposition of both alumina and ZnO.

Experimental

Substrates

The small silicon shards used in this study (ca. 1 cm×1 cm) were cut from 4″ wafers (University Wafer, South Boston, Mass.). The fused silica slides (1″×2″×1 mm) were purchased from Ted Pella (Redding, Calif.). These silicon and fused silica substrates were stored at room temperature and atmospheric pressure, and were cleaned before ALD and thermal evaporation.

Sample Cleaning

Prior to salt deposition, substrates were cleaned for one minute in an air plasma in a Model No. PDC-32G plasma cleaner (Harrick Plasma, Ithaca, N.Y.), or cleaned for 40 min in piranha solution (a ca. 7:3 mixture of H₂SO₄ (conc.) and 30% H₂O₂) at 80-100° C. After piranha cleaning, the substrates were washed extensively with high purity (18 MSΩ) water.

Thermal Deposition

Sodium chloride was evaporated using a DV-502A deposition system from Denton Vacuum (Moorestown, N.J.). Depositions of ca. 100 nm took about 10 min. The system had a rotating sample stage to improve film uniformity, an Inficon quartz-crystal thickness monitor (QCM), and a shutter activated by the QCM to precisely control the film thickness. Depositions ceased when the QCM thickness reached the desired value set at 10, 50, or 100 nm, where the QCM had previously been calibrated and the density of NaCl inputted into it. Fused silica slides and/or silicon wafers were mounted on the rotating platform of the system with vacuum tape. A small amount of sodium chloride (approx. 100 mg) was place in an aluminum boat connected to two electrodes. The system was then pumped to high vacuum (10⁻⁵-10⁻⁷ torr). During the deposition, the platform was rotated at 100 rpm to ensure uniform salt deposition on the substrate.

Atomic Layer Deposition

ALD of alumina was performed with a Kurt J. Lesker (Jefferson Hills, Pa.) ALD-150LX system. The precursors used for alumina deposition were trimethylaluminum (TMA) and water. Our ALD instrument is equipped with an in-situ FS-1® ellipsometer (FilmSense, Lincoln, Nebr.) that measures the thickness of ALD alumina films during a deposition. The deposition of Al₂O₃ followed the manufacturer's recommended recipe as follows. The ALD chamber was heated to 332° C. prior to initiation of the deposition, and this temperature was maintained during the deposition. The dose times for TMA and water were 21.0 ms and 15.5 ms, respectively, with 15,000 ms purge times for both precursors. The precursors used for zinc oxide deposition were diethylzinc (DEZ) and water. Dose times for DEZ and water were 21.0 ms and 15.5 ms, respectively, with 15,000 ms purge times for both precursors. For deposition of zinc oxide, 100 ALD cycles were used and the deposition temperature was 200° C.

Spectroscopic Ellipsometry (SE)

Spectroscopic ellipsometry was performed using a J. A. Woollam (Lincoln, Nebr.) M-2000DI ellipsometer over a wavelength range of 191-1688 nm. This ellipsometer can collect data at different angles and is equipped with a CCD array detector, a rotating compensator, and a near IR extension (out to 1688 nm).

X-Ray Photoelectron Spectroscopy (XPS)

X-ray photoelectron spectroscopy (XPS) was performed with a Surface Science SSX-100 X-ray photoelectron spectrometer (serviced by Service Physics, Bend, Oreg.) with a monochromatic Al K_{α} source, a hemispherical analyzer, and a take-off angle of 35°. Survey scans were recorded with a spot size of 800 μm×800 μm and a resolution of 4 (nominal pass energy of 150.0 eV). An electron flood gun for charge compensation was employed for XPS measurements. XPS peaks were referenced to the C is hydrocarbon signal (taken at 285.0 eV) when sample charging was observed. While this method is less than ideal, it is adequate to allow peak identification.

Removal of Sodium Chloride

Sodium chloride coatings were removed by sonicating three times in high purity water for 5 min, where the water was replaced after each sonication. Care was taken in cleaning the glassware for this work and also the tweezers that held the substrates.

Sodium Chloride Deposition for Surface Protection

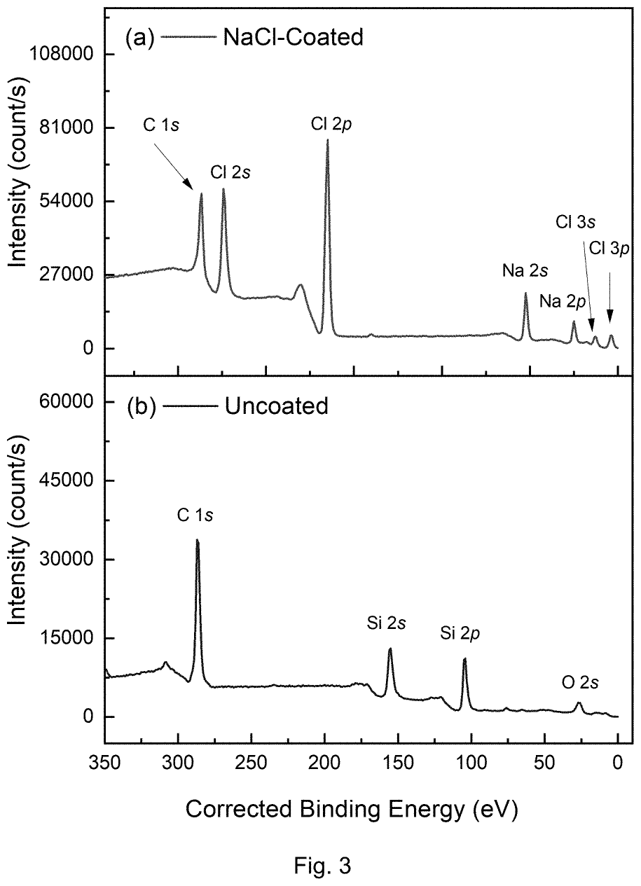

To determine the most effective barrier layer for protecting silicon wafers from contamination and preventing ALD, three different thicknesses of sodium chloride (nominal/QCM thicknesses of 10, 50, and 100 nm) were evaporated onto silicon and/or fused silica substrates. As expected, deposition of a ca. 100 nm transparent film of NaCl on the silicon wafers changed their apparent color from grey to a blueish-purple hue. By eye, these depositions (the color across the silicon surface) were uniform. There was no change in the appearance of the transparent fused silica slides after NaCl deposition. The presence of the NaCl films was further confirmed by X-ray photoelectron spectroscopy (XPS) and spectroscopic ellipsometry (SE). FIG. 3 (a. upper graph) shows XPS of a fused silica surface that was coated on one side with ca. 100 nm of NaCl. As expected, the NaCl-coated side shows only peaks attributable to Na and Cl (Na 2s and Na 2p signals at 64.0 eV and 31.0 eV, respectively, and Cl 2s and Cl 2p signals 271.0 eV and 200.0 eV, respectively), and adventitious carbon. In contrast, FIG. 3 (b. lower graph) is the XPS of the uncoated side of the fused silica substrate, which shows no Na or Cl—only Si, 0, and C. The absence of substrate signals from the NaCl-coated side of the substrate is consistent with an NaCl film that is without pinholes and at least 10 nm thick (XPS probes 5-10 nm into materials).

As a second example, an NaCl film (QCM thickness of 100 nm) was evaporated onto a piece of a silicon wafer. It was then analyzed by SE, where the film and substrate were modeled as the silicon substrate, a layer of native oxide (1.6 nm, as measured before the NaCl deposition), an NaCl film, and a roughness layer (a Bruggeman effective medium approximation layer based on a 50:50 mixture of void (air) and NaCl). The optical constants from the instrument software were used for all the layers (Si, native oxide, and NaCl), where the NaCl optical constants were based on a Sellmeier dispersion model. This model produced a fit with an NaCl film thickness of 106.9 nm, a roughness of 4.7 nm, and a reasonable mean squared error (MSE) value of 5.7. Uniqueness plots for the fit, based on the film thickness and roughness, were generated. The resulting ‘V’ or ‘U’ shapes suggested that the fit parameters were not correlated. Allowing the parameters in the NaCl Sellmeier model to vary or introducing thickness non-uniformity into the model did not significantly improve the quality of the fit or change the resulting thickness (these fits were also unique). Thermal salt deposition in our system was moderately uniform. In the case of a different ca. 100 nm salt film deposited over a 4″ silicon wafer, the thickness was 98.5±4.3 nm (average and standard deviation of 10 measurements), where the maximum and minimum thicknesses measured by SE over the wafer were 104.7 nm and 91.8 nm. In contrast, our ALD film deposition was much more uniform. For example, after 100 cycles of TMA and water, the thickness of an Al₂O₃ film over a 4″ silicon wafer was 8.4±0.1 nm (average and standard deviation of 10 measurements), where the maximum and minimum thicknesses measured here were 8.3 nm and 8.5 nm.

Substrate Protection with Evaporated NaCl

To test the ability of a removable salt coating to protect a silicon wafer from contamination, plasma cleaned silicon surfaces were coated with ca. 100 nm of NaCl, where the thicknesses and chemistries of these films were confirmed by SE and/or XPS (see above). The NaCl-coated surfaces were then exposed to the laboratory environment for 1, 3, and 7 months. They were then rinsed with water to remove the NaCl barrier layer, their advancing water contact angles were measured, and 100 cycles of ALD alumina from TMA and water were deposited on them. This deposition of alumina was used to test the availability/accessibility of the surface silanols, i.e., it was expected that a contaminated surface would show less reactivity than a clean one. Table 1 shows the results from these experiments. The first four rows of the table demonstrate that there is no statistical difference between the surface that was cleaned and immediately coated with ALD alumina and those that were coated with NaCl, exposed to the laboratory environment for extended periods of time, rinsed (deprotected), and coated with ALD alumina. As a control experiment, a silicon wafer was plasma cleaned, not coated with NaCl or anything else, and exposed to the laboratory environment for 67 days. After rinsing with water, its contact angle was noticeably higher than those of the pristine or NaCl-protected and deprotected silicon surfaces. The ALD film of alumina on this surface is also noticeably thinner and less uniform (the standard deviation is higher). These results suggest that removable salt coatings keep cleaned silicon wafers in their pristine state for extended periods of time.

TABLE 1
Experimental data for NaCl-coated and uncoated silicon shards after
exposure to the laboratory environment and ALD of alumina.

| Sample (Coated or | Time surface | | | Advancing | |
|---|---|---|---|---|---|
| Uncoated with ca. | exposed to the | | Increase in | water | Thickness of |
| 100 nm NaCl after | laboratory | | apparent SiO₂ | contact | alumina after 100 |
| plasma cleaning) | environment | | thickness* | angle* | ALD cycles*^{,†} |
| Uncoated | 0 | days | 0.08 ± 0.03 nm | <10° | 8.4 ± 0.1 nm |
| Coated | 1 | mo. | 0.07 ± 0.02 nm | <10° | 8.4 ± 0.1 nm |
| Coated | 3 | mo. | 0.10 ± 0.03 nm | <10° | 8.3 ± 0.1 nm |
| Coated | 7 | mo. | 0.07 ± 0.03 nm | <10° | 8.5 ± 0.1 nm |
| Uncoated | 67 | days | 0.15 ± 0.03 nm | 35° | 7.5 ± 0.3 nm |
| | | | | (After water | |
| | | | | treatment) | |

*After exposure to the lab and water wash.
^{†}Averages and standard deviations of three measurements on one sample.

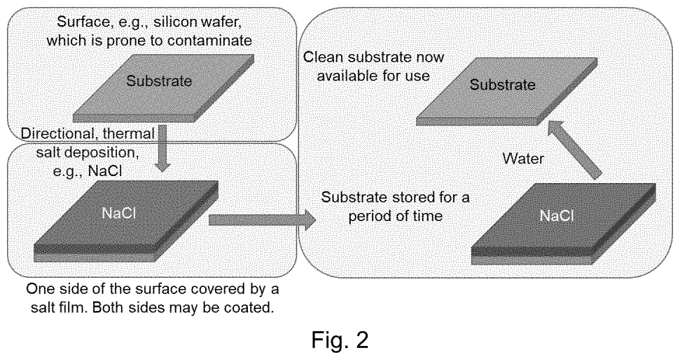

FIG. 2 discloses schematic flow diagram for a method for protecting and deprotecting surfaces with removable salt coatings. In the process, a removable salt coating is deposited on a substrate surface prone to contaminate, the coated substrate is stored for a period of time, and then the salt coating is removed to reveal the substrate surface available for use.

ALD on Salt-Protected Substrates and Deprotection of these Surfaces

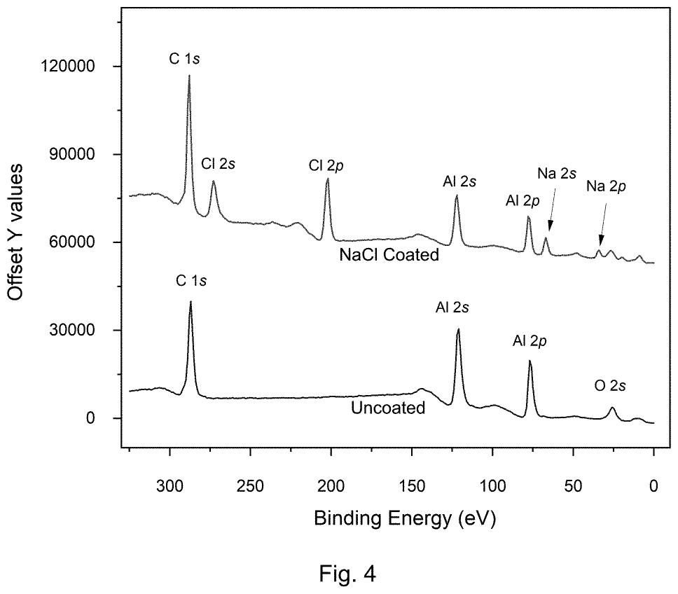

To test the ability of salt-coated surfaces to prevent ALD deposition on the underlying substrate, NaCl-coated fused silica substrates were placed in the ALD tool, with the uncoated surface face up, and alumina was deposited via 100 cycles of TMA and water. FIG. 4 shows the resulting XPS spectra. Rather strong Al 2s and 2p signals are clearly visible on both the ‘NaCl-coated’ and ‘uncoated’ surfaces, although the spectrum from the ‘NaCl-coated’ surface also contains signals from Na and Cl. Clearly, the alumina film on the ‘NaCl-coated’ surface of the substrate was not thick enough to obscure the signals from the salt and/or it is patchy/incomplete. Obviously, these results are a manifestation of ALD's lack of directionality. TMA may react with the NaCl film via water that may have been present in it before the deposition, or that is introduced during ALD.

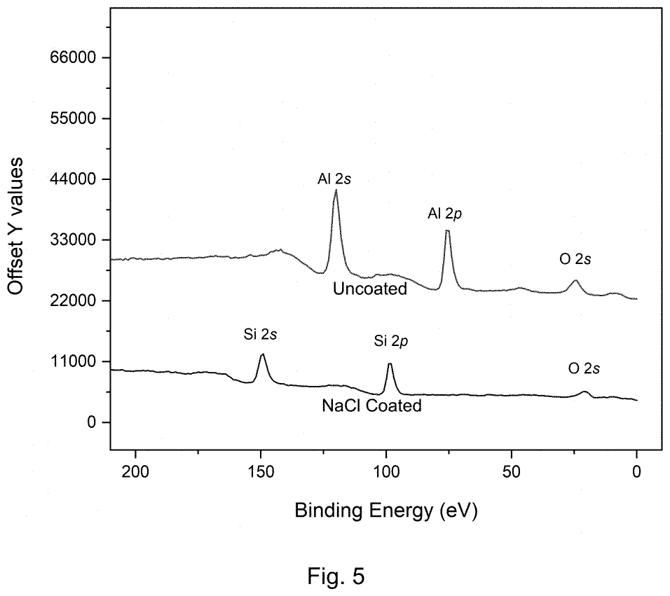

Removal of the NaCl coating on the alumina-coated fused silica slide was accomplished by sonicating/rinsing with water. This process removed unwanted alumina deposition on the backside of the surface. For example, FIG. 5 shows Al 2s and 2p XPS signals from the front side of the substrate, which was not coated with NaCl, while only the substrate signals (Si 2s and 2p), and no peaks from Na, Cl, or Al are observed on the backside of the slide. SE similarly confirmed the complete removal of salt films after sonication/rinsing.

Effect of Salt Thickness on Deprotection

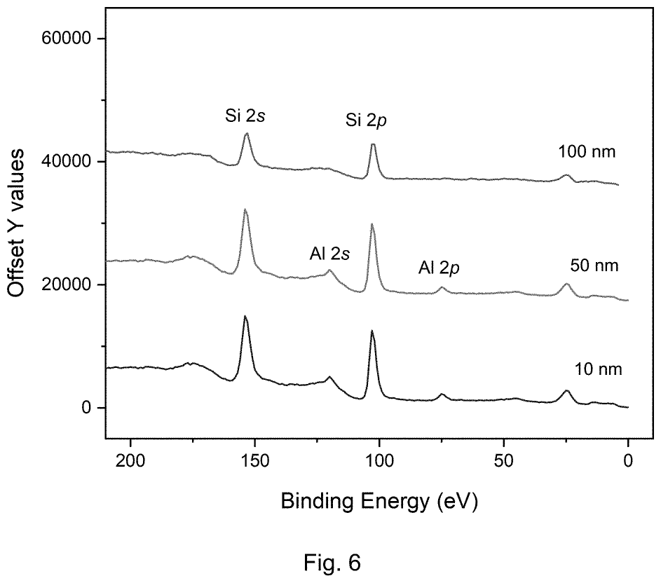

Different thicknesses of NaCl (10, 50 and 100 nm) were evaporated onto one side of fused silica substrates to test their ability to direct/limit ALD deposition. After ALD of Al₂O₃ on the surfaces and sonication/rinsing, these substrates were analyzed by XPS. As shown in FIG. 6, small aluminum signals were present on the fused silica slides that had previously been coated with 10 and 50 nm of NaCl. However, no aluminum signals were observed on the surface that was coated with 100 nm of NaCl. That is, 100 nm of NaCl appears to be an adequate barrier layer to prevent Al₂O₃ ALD deposition.

ALD of Zinc Oxide on NaCl-Protected Fused Silica

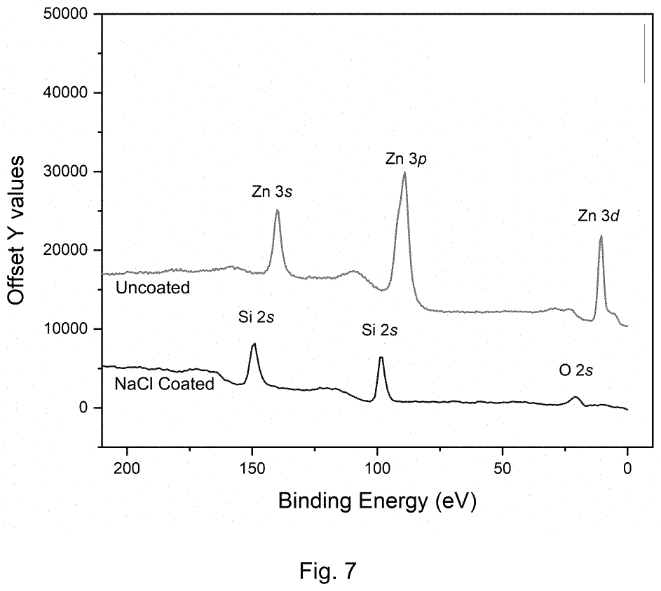

To test the generality of the disclosed invention, fused silica was coated on one side with ca. 100 nm of NaCl, after which the material was coated with ZnO by ALD via 100 cycles of diethylzinc and water, and then sonicated/rinsed with water. After this deprotection, Zn is only present on the side of the substrate that was originally unprotected (see FIG. 7).

The disclosed example demonstrated a method for protecting surfaces from unwanted contamination and ALD deposition using thin sodium chloride coating. This process employs thermal evaporation as a directional coating method and ALD as a non-directional one. The approach coated a surface with an evaporated salt coating to prevent environmental contamination or coated one side of a substrate with a salt film to prevent unwanted ALD deposition. The salt film was easily removed by sonication/rinsing in water. Moderately thick NaCl films (100 nm) effectively directed ALD deposition of Al₂O₃ and ZnO and prevented surface contamination. Results of area-selective depositions and surface protection were confirmed by XPS and SE.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A method for preventing contamination of a substrate surface comprising:
obtaining a substrate having a surface to be protected from contamination; and
depositing a removable protective salt coating on the substrate surface.

2. The method for preventing surface contamination of a substrate surface according to claim 1, wherein the substrate surface comprises a thiol-on-gold monolayer.

3. The method for preventing surface contamination of a substrate surface according to claim 2, wherein the thiol-on-gold monolayer surface comprises an alkyl thiol.

4. The method for preventing surface contamination of a substrate surface according to claim 2, wherein the thiol-on-gold monolayer surface comprises a thiol connected to a biologically active molecule.

5. The method for preventing surface contamination of a substrate surface according to claim 1, wherein the substrate surface comprises a chemically derivatized surface comprising a coating of silane molecules derivatized with biologically active molecules.

6. The method for preventing surface contamination of a substrate surface according to claim 1, wherein the substrate is a silicon wafer.

7. The method for preventing surface contamination of a substrate surface according to claim 1, wherein the substrate is glass.

8. The method for preventing surface contamination of a substrate surface according to claim 1, wherein the substrate is a dental crown or dental implant.

9. The method for preventing surface contamination of a substrate surface according to claim 1, wherein the protective salt coating has a thickness in the range of 50 nm to 1 μm.

10. The method for preventing surface contamination of a substrate surface according to claim 1, further comprising storing the substrate surface having the removable protective salt coating for a time period.

11. The method for preventing surface contamination of a substrate surface according to claim 10, further comprising removing the protective salt coating by washing the substrate surface.

12. A method for selectively preventing atomic layer deposition (ALD) on a substrate surface exposed to an ALD process, comprising:
depositing a removable protective salt coating on the substrate surface;
exposing the surface to an ALD process; and
removing the protective salt coating.

13. The method for selectively preventing ALD on a substrate according to claim 12, wherein the protective salt coating comprises a water-soluble inorganic salt.

14. The method for selectively preventing ALD on a substrate according to claim 12, wherein the protective salt coating is deposited by thermal evaporation.

15. The method for selectively preventing ALD on a substrate according to claim 12, wherein the protective salt coating has a thickness in the range of 50 nm to 1 μm.

16. A thiol-on-gold monolayer surface protected from contamination comprising:
a substrate surface comprising a thiol-on-gold monolayer; and
a removable salt coating deposited on the thiol-on-gold monolayer, wherein the salt coating has a thickness in the range of 50 nm to 1 μm.

17. The thiol-on-gold monolayer surface protected from contamination according to claim 16, wherein the protective salt coating comprises a water-soluble inorganic salt.

18. The thiol-on-gold monolayer surface protected from contamination according to claim 16, wherein the thiol-on-gold monolayer surface is included in a biosensor.

19. The thiol-on-gold monolayer surface protected from contamination according to claim 16, wherein the thiol-on-gold monolayer surface comprises an alkyl thiol.

20. The thiol-on-gold monolayer surface protected from contamination according to claim 16, wherein the thiol-on-gold monolayer surface comprises a thiol connected to a biologically active molecule.
